(19) United States
(12) Patent Application Publication
(10) Pub. No.: US 2025/0353959 A1
(43) Pub. Date: Nov. 20, 2025

(54) PREPARATION METHOD FOR LOW-COLOR NUMBER, LOW-ODOR POLYISOCYANATE CURING AGENT

(71) Applicant: WANHUA CHEMICAL GROUP CO., LTD, Yantai, Shandong (CN)

(72) Inventors: Wei LIU, Yantai (CN); Yonghua SHANG, Yantai (CN); Chengdong LIN, Yantai (CN); Shuchang SUN, Yantai (CN); Dan WANG, Yantai (CN); Shaohui XIN, Yantai (CN); Zhenmin YANG, Yantai (CN); Mingjie LI, Yantai (CN); Ying CHENG, Yantai (CN); Yuan LI, Yantai (CN)

(73) Assignee: WANHUA CHEMICAL GROUP CO., LTD, Yantai, Shandong (CN)

(21) Appl. No.: 18/869,249

(22) PCT Filed: Oct. 27, 2023
(86) PCT No.: PCT/CN2023/127304

§ 371 Date: Nov. 25, 2024

(30) Foreign Application Priority Data
Oct. 28, 2022 (CN) 202211336745.6

(51) Int. Cl. C08G 71/04

(52) U.S. Cl. CPC C08G 71/04

(57) ABSTRACT

Disclosed is a preparation method for a low-color number, low-odor polyisocyanate curing agent. The preparation method comprises a step of carrying out a polymerization reaction on a diisocyanate monomer under the action of a trimerization catalyst in a polymerization reaction kettle with continuously flowing inert gas, an upper head of the polymerization reaction kettle being provided with an inert gas inlet pipe, the inert gas inlet pipe being an insertion pipe, and the upper head of the polymerization reaction kettle also being provided with an inert gas outlet and a central stirring shaft; the insertion opening position (A) of the inert gas inlet pipe and the inert gas outlet position (B) on the surface of the upper head form an angle α (∠ACB) with the fixed position (C) of the central stirring shaft projected onto the plane on the upper head surface, where 30°≤α≤180°. The present application can achieve stable catalytic activity of a catalyst by means of controlling the use of nitrogen during the reaction process, which is conducive to stable process control, resulting in a low color number and reduced amine odor in the obtained product.

### FIELD OF THE INVENTION

Embodiments of the present application relate to a method for preparing a light-colored, low-odor polyisocyanate curing agent, which is mainly used in the field of polyurethane coatings or adhesives.

### BACKGROUND OF THE INVENTION

Isocyanurate, i.e. the six-membered ring structure generated by the trimerization reaction of isocyanate monomers. Polyisocyanates with this structure have good thermal stability, especially polyisocyanate curing agents of aliphatic or alicyclic isocyanates, which are widely used curing agent products in the field of coatings and adhesives.

Techniques for modifying diisocyanate monomers in the presence of catalysts are well known in the art, for example, performing trimerization reactions or alcohol modification reactions, and then using vacuum distillation or thin film evaporation to remove unreacted monomers after reaching the desired conversion rate, thereby obtaining polyisocyanate curing agent products, as can be seen in U.S. Pat. Nos. 4,288,586, 6,093,817, CN107827832, EP0330966A2, and the like.

However, in the actual industrial implementation process, during the conventional nitrogen protection process, it is found that the catalyst in the reaction process has unstable catalytic activity, which in turn leads to problems such as excessive catalyst amount, unstable product quality, for example, the color number of the product is elevated, and due to the residual catalyst decomposition products in the products, an amine odor is easily appeared in the process of use of the products, which affects the downstream customer's experience of using the products.

Therefore, it is of great significance for industrial production to develop a simple and practical method for preparing a stable polyisocyanate curing agent with low color number and low odor.

### SUMMARY OF THE INVENTION

The following is a summary of the subject matter described in detail herein. This summary is not intended to limit the scope of protection of the claims.

In the related technology, isocyanate is usually subjected to catalytic polymerization reaction in an inert gas environment, such as the protection of nitrogen or the protection of nitrogen under the condition of circulation, which is a conventional operation in the field, and there is no special requirement for the specific operation mode of the inert gas protection in the presently disclosed technologies. However, during the experimental process of the catalytic polymerization reaction of diisocyanate, the researchers of the present application were surprised to find that the way of inert gas circulation in the polymerization reaction kettle can affect the activity of the trimerization catalyst, which in turn affects the control of the reaction process and the quality of the final product, such as the color number and odor. The applicant speculated that the reason may be that the catalyst will form catalyst poisons after dispersing in the isocyanate system, while the continuous inert gas blowing and disturbing to the liquid level of the isocyanate material on the one hand may bring out part of the poisons, on the other hand, it can avoid the combination of poisons with the catalyst to a certain extent, so as to ensure the catalytic activity of the catalyst effectively. At the same time, the larger the area of isocyanate liquid level blown by inert gas in the polymerization reaction kettle, the more favorable to the catalyst activity, on the contrary, if the effect of liquid level blowing is not achieved, it will be unfavorable to the catalyst activity.

In order to achieve the above purpose, the applicant of the present application has regulated the specific layout and implementation conditions of the inert gas through a large number of experiments, and finally developed a method for preparing a light-colored, low-odor polyisocyanate curing agent by controlling the layout of the position of the inert gas and the pressure in the polymerization reaction kettle.

The technical solution of the embodiments of the present application is as follows.

The present application provides a method for preparing a light-colored, low-odor polyisocyanate curing agent, comprising a step of carrying out a polymerization reaction on diisocyanate monomers under the action of a trimerization catalyst in a polymerization reaction kettle with continuously flowing inert gas, wherein an upper head of the polymerization reaction kettle is provided with an inert gas inlet pipeline, the inert gas inlet pipeline is an insertion pipe, and the upper head of the polymerization reaction kettle is also provided with an inert gas outlet and a central stirring shaft; and
an angle α (∠ACB) is formed by projecting lines connecting insertion pipe opening position (A) of the inert gas inlet pipeline and inert gas outlet position (B) on the surface of the upper head with fixed position (C) of the central stirring shaft on the surface of the upper head respectively onto a plane, wherein 30°≤α (∠ACB) ≤180°, preferably 90°≤α (∠ACB)≤180°, more preferably 150°≤α (∠ACB)≤180°, and the plane is formed by looking down from the top of the polymerization reaction kettle.

In the present application, during continuous flow of the inert gas within the polymerization reaction kettle, the inert gas is controlled at a pressure of 1 to 100 kPaG, preferably 2 to 60 kPaG, and more preferably 3 to 30 kPaG. If the pressure is too high, the requirements to the connection among the polymerization reaction kettle itself and the various valves are high, while if the pressure is too low, it will be very unfavorable to the isocyanate catalytic reaction. During the polymerization reaction, the present application requires the inert gas in the polymerization reaction kettle to be in a state of constant circulation, in which the adjustment of the pressure in the kettle can be achieved by controlling the gas inventory in the kettle and other conventional means in the field, for example, by specifically adjusting the opening degree of the inert gas inlet or outlet valves.

In the present application, the inert gas is introduced into the polymerization reaction kettle from the insertion pipe opening position of the inert gas inlet pipeline, and the flow rate of the gas at the inlet is in a range from 0.05 m/s to 60 m/s, preferably in a range from 0.5 m/s to 30 m/s; and
the direction and number of openings of the insertion pipe in the polymerization reaction kettle are not specifically limited, but according to experimental results, it is preferred that the opening of the insertion pipe in the polymerization reaction kettle is orientated in such a way that the opening is vertically downward or is inclined at an angle of less than 30°, and it is more preferred that the opening is vertically downward; and the number of openings of the insertion pipe can be one, two or more.

In the present application, the vertical distance between the inert gas inlet pipeline and the central stirring shaft is 0.2 to 1 times the radius of the polymerization reaction kettle, such as 0.4, 0.6, and 0.8 times; and the vertical distance between the inert gas outlet and the central stirring shaft is 0.2 to 1 times the radius of the polymerization reaction kettle, such as 0.4, 0.6, and 0.8 times. By controlling the positions of the inlet and outlet of the inert gas, the present application is able to increase as much as possible the area through which the inert gas blowing process passes under the premise of ensuring the angle of each opening position, which is conducive to maintaining the catalytic activity of the catalyst. When the blowing area is too small, it is not conducive to the maintenance of catalytic activity of the catalyst, but it is preferable to keep the inert gas inlet pipeline and outlet pipeline at an appropriate distance from the edge of the head; and the closer the distance, the more unfavorable it is for construction and the more likely there are safety hazards.

In the present application, the inert gas inlet pipeline is an insertion pipe, the insertion length of the insertion pipe in the polymerization reaction kettle is not particularly limited, and can be adjusted based on the size of the polymerization reaction kettle and the filling rate of the material, but according to the experimental effect, it is shown that in some specific examples, preferably, the insertion length of the insertion pipe in the polymerization reaction kettle is in a range from 5 cm to 50 cm, preferably in a range from 10 cm to 30 cm; and
the insertion pipe opening position of the insertion pipe can be located above or below the liquid level of the material, there is no specific limitation in the present application, but according to the experimental effect, it is preferably to be above the liquid level of the material in the polymerization reaction kettle, and more preferably, the insertion pipe opening position is located 5 to 50 cm, preferably 20 to 30 cm, above the liquid level of the material. The distance between the insertion pipe opening position of the inert gas inlet pipeline and the liquid level will affect the degree of disturbance between the inert gas and the isocyanate liquid level; the closer the distance, the stronger the disturbance and the more favorable it will be, but if the distance is too close, it may lead to splashing of isocyanate material into the pipe inlet, and the pipe will be easily clogged after a long period of operation.

In the present application, the inert gas is one or more selected from the group consisting of helium gas, neon gas, argon gas, krypton gas and nitrogen gas, preferably argon gas and/or nitrogen gas.

In the present application, the shape of the upper head of the polymerization reaction kettle is not particularly limited, and any type commonly used in industrialized reaction kettle can be used, such as hemispherical, oval, butterfly and the like.

In the present application, the reaction route for preparing a polyisocyanate curing agent by polymerization of diisocyanate monomers under the action of a trimerization catalyst is an existing process, and the those skilled may, in accordance with the relevant technology, select the diisocyanate monomers, the trimerization catalyst, and the operating parameters of the reaction process, etc., for example, in some specific examples enumerated in the present application, the solution as described below may be adopted.

In the present application, the diisocyanate is one or more selected from the group consisting of aliphatic diisocyanates and alicyclic diisocyanates; preferably, the diisocyanate is one or more selected from the group consisting of tetramethylene diisocyanate, pentamethylene diisocyanate, hexamethylene diisocyanate, isophorone diisocyanate, dicyclohexylmethane diisocyanate, xylylene diisocyanate, cyclohexyldimethylene diisocyanate, and lysine diisocyanate; and more preferably hexamethylene diisocyanate and/or isophorone diisocyanate.

In the present application, the trimerization catalyst is one or more selected from the group consisting of weak acid salts of organic ammonium and metal salts of alkyl carboxylic acids; preferably, the trimerization catalyst is one or more selected from the group consisting of tetramethylammonium acetate, tetraethylammonium acetate, tetrabutylammonium acetate, dodecyltrimethylammonium octanoate, 2-hydroxy-N,N,N-trimethyl-1-propanaminium formate, 2-ethylhexanoic acid-N-(2-hydroxypropyl)-N,N,N-trimethylammonium salt, potassium acetate, potassium octoate, and lead 2-butylhexanoate; and more preferably, the trimerization catalyst is one or more selected from the group consisting of 2-hydroxy-N,N,N-trimethyl-1-propanaminium formate, 2-ethylhexanoic acid-N-(2-hydroxypropyl)-N,N,N-trimethylammonium salt, tetramethylammonium hydroxide, tetrabutylammonium hydroxide, tetraethylammonium hydroxide, and benzyltrimethylammonium hydroxide.

In the present application, the trimerization catalyst can be used in the absence of a solvent or can be dissolved in a solvent and used in the form of a solution; and
in some embodiments, the solvent is selected from the group consisting of straight or branched monohydric alcohols and/or dihydric alcohols containing 1-20 carbon atoms; or the solvent is selected from the group consisting of straight or branched alcohols containing 1-20carbon atoms, more than one hydroxyl group and optionally other heteroatom, wherein the heteroatom preferably is oxygen; and preferably, the solvent for dissolving the trimerization catalyst includes, but is not limited to, one or more of methanol, ethanol, 1-propanol, 2-propanol, n-butanol, i-butanol, s-butanol, t-butanol, n-octanol, i-octanol, heptanol, 2-ethyl-1,3-hexanediol, 1,3-butanediol, 1,4-butanediol, and 1-methoxy-2-propanol, and preferably one or more of ethanol, n-butanol, hexanol, heptanol, and i-octanol.

In some embodiments, when the trimerization catalyst is used in the form of a solution, the concentration of the trimerization catalyst solution is in a range from 5 wt % to 50 wt %, and preferably in a range from 10 wt % to 30 wt %.

In the present application, the amount of the tripolymerisation catalyst is 20 ppm to 500 ppm, preferably 50 ppm to 250 ppm, of the mass of the diisocyanate monomer, and the tripolymerisation catalyst can be added dropwise or all at once.

In the present application, the polymerization reaction is performed under the following conditions: a reaction temperature of 40° C. to 90° C., preferably 50° C. to 75° C., and a reaction time of 4 h to 20 h, preferably 5 h to 10 h.

In the present application, the method further comprises a step of terminating the reaction to obtain a reaction solution and a step of removing unreacted diisocyanate monomer from the reaction solution after completion of the polymerization reaction.

In some embodiments, the terminating of the reaction is performed when the conversion rate of the diisocyanate monomer reaches 20% to 70%, preferably 25% to 50%, and the conversion rate can be determined by monitoring the NCO content of the reaction system;
preferably, the terminating of the reaction is performed by deactivating the catalyst by adding an acidic substance;
the acidic substance is preferably one or more of hydrochloric acid, sulphuric acid, phosphoric acid, dibutyl phosphate, diisooctyl phosphate, and p-toluenesulfonic acid;
the acidic substance is added in an amount of 1 to 10 times, preferably 1.1 to 5 times the molar amount of catalyst;
alternatively, the terminating of the reaction is performed by thermal inactivating with a residence time of 15 min to 45 min at a temperature of 110° C. to 150° C., for example, with a residence time of 30 min at a temperature of 130° C.

In some embodiments, the removing of the unreacted diisocyanate monomer is carried out by means of evaporation method, and the evaporation method is any one selected from the group consisting of thin film evaporation method, falling film evaporation method, short range evaporation method, and reduced pressure rectification method. For example, if the thin film evaporation method is used, separation temperature is 140° C. to 200° C., the pressure is 1 PaG to 500 PaG, and a polyisocyanate product with a monomer content of less than 0.5 wt % and a color number of not more than 25 Hazen is obtained.

The polyisocyanate curing agent described in the present application can also be dissolved into a solvent to form a solution product, preferably at a concentration of 50-80 wt %; and
the solvent is selected from any one of butyl acetate, ethyl acetate, solvent oil, toluene, xylene, propylene glycol methyl ether acetate, diheptanone and the like, or a combination of at least two of them.

Compared with the related technology, the technical solution of the embodiments of the present application has the following beneficial effects:
the embodiments of the present application improve the stability of the catalytic activity of the catalyst by controlling the layout of the position of the inert gas and the pressure thereof in the polymerization reaction kettle during the polymerization reaction of the diisocyanate monomers, thereby achieving stable control of the reaction process, and obtaining a product with a light-colored, a low amine odor, and a good stability of product quality.

Other aspects can be understood after reading and understanding the accompanying drawings and detailed description.

### DESCRIPTION OF THE DRAWINGS

The accompanying drawings are used to provide a further understanding of the technical solutions herein and form part of the specification and are used in conjunction with the examples of the present application to explain the technical solutions herein and do not constitute a limitation of the technical solutions herein.

FIG. 1 shows a plan view of upper head layout of the polymerization reaction kettle of the present application viewed from the top of the polymerization reaction kettle.

In FIG. 1, A represents the insertion pipe opening position of the inert gas inlet pipeline, B represents the inert gas outlet position, C represents fixed position of the central stirring shaft, and a represents the angle formed by projecting (∠ACB);

FIG. 2 shows a central sectional view of the polymerization reaction kettle along the insertion pipe of the inert gas inlet pipeline of the present application.

In FIG. 2, A represents the insertion pipe opening position of the inert gas inlet pipeline, C represents fixed position of the central stirring shaft, and D represents the distance between the insertion pipe opening position and the liquid level of the material in the polymerization reaction kettle.

### DETAILED DESCRIPTION OF THE INVENTION

The methods provided by the present application will be further illustrated by the following examples, but the present application is not limited in any way thereby.

1. The source information of main raw materials are as follows, and the others without special instructions are ordinary commercially available raw materials.
Hexamethylene diisocyanate (HDI): WANHUA CHEMICAL GROUP CO., LTD;
Isophorone diisocyanate (IPDI): WANHUA CHEMICAL GROUP CO., LTD;
Pentamethylene diisocyanate (PDI): Mitsui Chemicals, Inc.;
Tetrabutylammonium acetate (catalyst a): sigma-Aldrich, 95%;
2-Ethylhexanoic acid-N-(2-hydroxypropyl)-N,N,N-trimethylammonium salt (catalyst b): AIR PRODUCTS, 97%;
Benzyltrimethylammonium hydroxide (catalyst c): sigma-Aldrich, 96%;
n-Hexanol: sigma-Aldrich, 98%;
n-Butanol: sigma-Aldrich, 98%;
n-Butyl acetate: Aladdin reagent platform.

2. The main test methods in the present application
1). Test of NCO content was carried out by adopting national standard GB/T 12009.4: The NCO group content based on the total mass of the sample was obtained by reverse titration using 1 mol/L hydrochloric acid after neutralizing the isocyanate group in the measured specimen with an excess of 2 mol/L di-n-butylamine.
2). Free isocyanate monomer content test: national standard GB/T 18446-2009 was used.
3). Color number test: BYK digital colorimeter (Germany BYK LCS IV) was used.
4). Evaluation method of catalyst activity: Based on isocyanate monomer, the temperature was maintained at 60° C., 150 ppm of catalyst was added at one time, and the rate of change of isocyanate content at the end of the reaction was compared:

Rate of change of isocyanate content=(theoretical content of isocyanate of initial isocyanate monomer−isocyanate content at the end of the reaction)/initial isocyanate content*100%;
where the higher rate of change indicates the higher activity, it is recommended to be higher than 30, more recommended to be higher than 35.
5). Evaluation method of amine odor of product: 400 g of product was placed into 500 ml of white small mouth bottle, sealed with a rubber stopper, and then it was placed in the 80° C. oven for heating for 2 h. After taking out the bottle, the rubber stopper was removed, and then one can gently stir it at the mouth of the bottle with hand to feel the grade of the amine odor, and classified into four levels: none, mild, slightly strong, and strong.

### EXAMPLES 1 TO 9

Preparation of trimerization catalyst solution: tetrabutylammonium acetate was dissolved in n-butanol to form a solution with a concentration of 20 wt %.

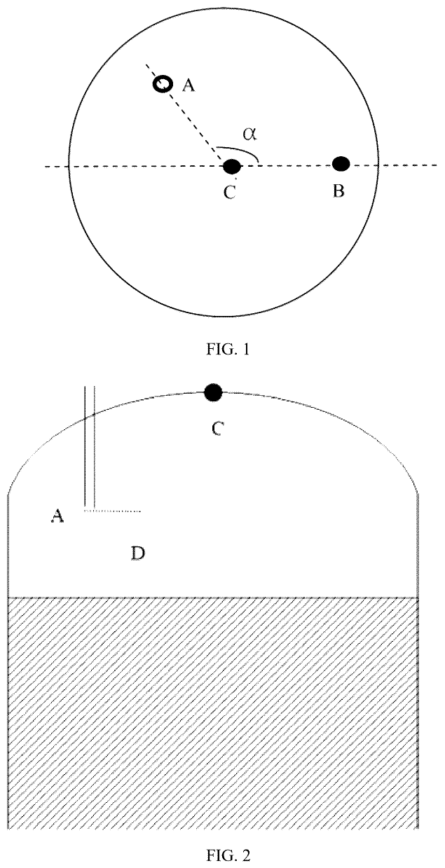

Preparation of the polymerization reaction kettle: an oval upper head was used, the polymerization reaction kettle was equipped with an inert gas inlet pipeline, a central stirring shaft and an inert gas outlet, and the layout of which was shown in FIGS. 1 and 2, wherein insertion pipe opening position (A) of the inert gas inlet pipeline, inert gas outlet position (B) on the surface of the upper head, and fixed position (C) of the central stirring shaft on the surface of the upper head are projected onto a plane to form an angle α (∠ACB), wherein 30°≤α≤180°.

The insertion length of the insertion pipe in the polymerization reaction kettle was 10 cm, the number of opening of the insertion pipe was 1, the opening position is located above the liquid level of the material in the polymerization reaction kettle, and the distance (recorded as D) is 5-50 cm.

The insertion pipe opening of the inert gas inlet pipeline is vertically downward, and the radius of the polymerization reaction kettle was recorded as R. The vertical distance (recorded as D1) between the insertion pipe of the inert gas inlet pipeline and the central stirring shaft is (0.2−1)×R; and the vertical distance (recorded as D2) between the inert gas outlet and the central stirring shaft is (0.2−1)×R.

The step of preparing the polyisocyanate curing agent was as follows.

Inert gas was continuously passed into the above polymerization reaction kettle and its flow rate and pressure were regulated, then 1,000 kg of diisocyanate monomer was placed in the polymerization reaction kettle, and the reaction system was heated up to 70° C. The above trimerization catalyst solution (the amount of the trimerization catalyst was 150 ppm of the mass of the diisocyanate monomer) was added dropwise into the reaction system under stirring. The polymerization reaction was carried out by controlling the reaction temperature to be between 70° C. and 80° C. When the conversion rate of diisocyanate was 60%, the reaction was terminated by adding dibutyl phosphate in an equimolar amount with the catalyst to obtain the polymerization reaction solution.

The unreacted diisocyanate monomer in the polymerization reaction solution was removed by evaporation using a thin-film evaporator at a temperature of 180° C. and an absolute pressure of 50 PaG, so that its content was less than 0.34 wt %, and then it was dissolved in butyl acetate to obtain a solution product with a concentration of 70 wt %. The main reaction conditions were shown in Table 1, and the results were shown in Table 2.

TABLE 1
Main parameters of the polymerization reaction
kettle and operation process of Examples 1-9

| | | | | | | | Parameters of inert gas | | |
|---|---|---|---|---|---|---|---|---|---|
| | Parameters of polymerization | | | | | | | Flow | Pressure |
| | reaction kettle | | | | | Diisocyanate | | rate/ | in kettle/ |
| | α/° | D/m | D1/R | D2/R | Catalyst | monomer | Species | (m/s) | KPaG |
| Example 1 | 30 | 10 | 0.2 | 0.2 | a | IPDI | N₂ | 60 | 80 |
| Example 2 | 45 | 15 | 0.3 | 0.35 | b | IPDI | N₂ | 40 | 100 |
| Example 3 | 60 | 5 | 0.4 | 0.4 | b | IPDI | N₂ | 20 | 60 |
| Example 4 | 90 | 20 | 0.5 | 0.5 | c | IPDI | N₂ | 10 | 40 |
| Example 5 | 120 | 30 | 0.7 | 0.6 | c | HDI | N₂ | 5 | 10 |
| Example 6 | 180 | 40 | 0.9 | 0.9 | c | IPDI | N₂ | 2 | 5 |
| Example 7 | 150 | 50 | 1 | 1 | c | PDI | N₂ | 1 | 3 |
| Example 8 | 30 | 10 | 0.2 | 0.2 | a | IPDI | N₂ | 0.05 | 1 |

### COMPARATIVE EXAMPLE 1

Referring to the method of Example 6, the difference was that the angle α (∠ACB) was adjusted to 25° in the polymerization reaction kettle, and other operating conditions remained unchanged, and the results are shown in Table 2.

### COMPARATIVE EXAMPLE 2

Referring to the method of Example 6, the difference was that the inert gas was continuously flowing in the polymerization reaction kettle, only the flow rate was adjusted to 0.04 m/s, other operating conditions remain unchanged, and the results are shown in Table 2.

### COMPARATIVE EXAMPLE 3

Referring to the method of Example 6, the difference was that nitrogen gas was not circulated. Only before the start of the polymerization reaction, nitrogen gas was passed into the polymerization reaction kettle for nitrogen gas replacement for protection, the pressure was 5 kPaG, then nitrogen gas was no longer passed in during the polymerization reaction, and the other operating conditions remain unchanged. The results were shown in Table 2.

### COMPARATIVE EXAMPLE 4

Referring to the method of Example 6, the difference was that the vertical distance D1 between the insertion pipe of the inert gas inlet pipeline and the central stirring shaft was adjusted to 0.15×R, and the other operating conditions remain unchanged. The results were shown in Table 2.

### COMPARATIVE EXAMPLE 5

Referring to the method of Example 6, the difference was that vertical distance D1 between the insertion pipe of the inert gas inlet pipeline and the central stirring shaft as well as vertical distance D2 between the outlet pipeline and the central stirring shaft were adjusted to 0.15×R at the same time, and the other operating conditions remain unchanged. The results were shown in Table 2.

### COMPARATIVE EXAMPLE 6

Referring to the method of Example 6, the difference was that vertical distance D2 between the inert gas outlet and the central stirring shaft was adjusted to 0.15×R, and the other operating conditions remain unchanged. The results were shown in Table 2.

TABLE 2
Comparison of catalyst activity, product color and
odor in the examples and the comparative examples

| | Catalyst activity | Curing agent product index | |
|---|---|---|---|
| | Rate of change of | Color number | amine |
| | isocyanate content/% | (Hazen) | odor |
| Example 1 | 31 | 25 | mild |
| Example 2 | 32 | 23 | mild |
| Example 3 | 33 | 18 | none |
| Example 4 | 35 | 15 | none |
| Example 5 | 36 | 14 | none |
| Example 6 | 40 | 14 | none |
| Example 7 | 32 | 20 | mild |
| Example 8 | 30 | 29 | mild |
| Comparative | 25 | 33 | slightly strong |
| Example 1 | | | |
| Comparative | 26 | 35 | slightly strong |
| Example 2 | | | |
| Comparative | 15 | 48 | strong |
| Example 3 | | | |
| Comparative | 22 | 39 | slightly strong |
| Example 4 | | | |
| Comparative | 18 | 44 | strong |
| Example 5 | | | |
| Comparative | 23 | 39 | slightly strong |
| Example 6 | | | |

From the data in Table 2 above, it can be seen that controlling the circulation of inert gas and the manner of its introduction can effectively maintain the catalyst activity and improve the quality of the product such as color number and odor.

1. A method for preparing a light-colored, low-odor polyisocyanate curing agent, comprising a step of carrying out a polymerization reaction on diisocyanate monomer under the action of trimerization catalyst in a polymerization reaction kettle in which inert gas is continuously flowing, wherein an upper head of the polymerization reaction kettle is provided with an inert gas inlet pipeline, the inert gas inlet pipeline is an insertion pipe, and the upper head of the polymerization reaction kettle is also provided with an inert gas outlet and a central stirring shaft;
an angle α (∠ACB) is formed by projecting lines connecting insertion pipe opening position (A) of the inert gas inlet pipeline and inert gas outlet position (B) on the surface of the upper head with fixed position (C) of the central stirring shaft on the surface of the upper head respectively onto a plane, wherein 30°≤α≤180°, and the plane is formed by looking down from the top of the polymerization reaction kettle.

2. The method of claim 1, wherein 90°≤α (∠ACB)≤180°, preferably 150°≤α (∠ACB)≤180°.

3. The method of claim 1, wherein, during a continuous flow of the inert gas within the polymerization reaction kettle, the inert gas is controlled at a pressure of 1 kPaG to 100 kPaG, preferably 2 kPaG to 60 kPaG, and more preferably 3 kPaG to 30 kPaG.

4. The method of claim 1, wherein the inert gas is introduced into the polymerization reaction kettle from the insertion pipe opening position of the inert gas inlet pipeline, and the flow rate of the gas at the inlet is in a range from 0.05 m/s to 60 m/s, preferably in a range from 0.5 m/s to 30 m/s;
the opening of the insertion pipe in the polymerization reaction kettle is orientated in such a way that the opening is vertically downward or is inclined at an angle of less than 30°, preferably the opening is vertically downward; and
the number of openings of the insertion pipe can be one, two, or more.

5. The method of claim 1, wherein the vertical distance between the inert gas inlet pipeline and the central stirring shaft is 0.2 to 1 times the radius of the polymerization reaction kettle; and the vertical distance between the inert gas outlet and the central stirring shaft is 0.2 to 1 times the radius of the polymerization reaction kettle.

6. The method of claim 1, wherein the insertion pipe opening position of the inert gas inlet pipeline can be located above or below the liquid level of the material, preferably above the liquid level of the material in the polymerization reaction kettle, and more preferably the insertion pipe opening position is located 5 to 50 cm, preferably 20 to 30 cm, above the liquid level of the material.

7. The method of claim 1, wherein the inert gas is one or more selected from the group consisting of helium gas, neon gas, argon gas, krypton gas and nitrogen gas, preferably argon gas and/or nitrogen gas.

8. The method of claim 1, wherein the diisocyanate is one or more selected from the group consisting of aliphatic diisocyanates and alicyclic diisocyanates.

9. The method of claim 8, wherein the diisocyanate is one or more selected from the group consisting of tetramethylene diisocyanate, pentamethylene diisocyanate, hexamethylene diisocyanate, isophorone diisocyanate, dicyclohexylmethane diisocyanate, xylylene diisocyanate, cyclohexyldimethylene diisocyanate, and lysine diisocyanate; and more preferably hexamethylene diisocyanate and/or isophorone diisocyanate.

10. The method of claim 1, wherein the trimerization catalyst is one or more selected from the group consisting of weak acid salts of organic ammonium and metal salts of alkyl carboxylic acids.

11. The method of claim 10, wherein the trimerization catalyst is one or more selected from the group consisting of tetramethylammonium acetate, tetraethylammonium acetate, tetrabutylammonium acetate, dodecyltrimethylammonium octanoate, 2-hydroxy-N,N,N-trimethyl-1-propanaminium formate, 2-ethylhexanoic acid-N-(2-hydroxypropyl)-N,N,N-trimethylammonium salt, potassium acetate, potassium octoate, and lead 2-butylhexanoate; and more preferably the trimerization catalyst is one or more selected from the group consisting of 2-hydroxy-N,N,N-trimethyl-1-propanaminium formate, 2-ethylhexanoic acid-N-(2-hydroxypropyl)-N,N,N-trimethylammonium salt, tetramethylammonium hydroxide, tetrabutylammonium hydroxide, tetraethylammonium hydroxide, and benzyltrimethylammonium hydroxide.

12. The method of claim 1, wherein, the trimerization catalyst can be used in the absence of a solvent or can be dissolved in a solvent and used in the form of a solution;
the solvent is selected from the group consisting of straight or branched monohydric alcohols and/or dihydric alcohols containing 1-20 carbon atoms; or the solvent is selected from the group consisting of straight or branched alcohols containing 1-20 carbon atoms, more than one hydroxyl group and optionally other heteroatom, wherein the heteroatom preferably is oxygen; and preferably, the solvent for dissolving the trimerization catalyst includes, but is not limited to, one or more of methanol, ethanol, 1-propanol, 2-propanol, n-butanol, i-butanol, s-butanol, t-butanol, n-octanol, i-octanol, heptanol, 2-ethyl-1,3-hexanediol, 1,3-butanediol, 1,4-butanediol, and 1-methoxy-2-propanol, and preferably one or more of ethanol, n-butanol, hexanol, heptanol, and i-octanol; and
when the trimerization catalyst is used in the form of a solution, the concentration of the trimerization catalyst solution is in a range from 5 wt % to 50 wt %, and preferably in a range from 10 wt % to 30 wt %.

13. The method of claim 1, wherein the amount of the trimerization catalyst is 20 ppm to 500 ppm, preferably 50 ppm to 250 ppm, of the mass of the diisocyanate monomer, and the trimerization catalyst can be added dropwise or all at once; and
the polymerization reaction is performed under the following conditions: a reaction temperature of 40° C. to 90° C., preferably 50° C. to 75° C., and a reaction time of 4 h to 20 h, preferably 5 h to 10 h.

14. The method of claim 1, further comprising a step of terminating the reaction to obtain a reaction solution after completion of the polymerization reaction; wherein the terminating of the reaction is performed when the conversion rate of the diisocyanate monomer reaches 20% to 70%, preferably 25% to 50%, and the conversion rate can be determined by monitoring the NCO content of the reaction system;
preferably, the terminating of the reaction is performed by deactivating the catalyst through adding an acidic substance;
the acidic substance is preferably one or more of hydrochloric acid, sulphuric acid, phosphoric acid, dibutyl phosphate, diisooctyl phosphate, and p-toluenesulfonic acid;
the acidic substance is added in an amount of 1 to 10 times, preferably 1.1 to 5 times the molar amount of catalyst; or
the terminating of the reaction is performed by thermal inactivating with a residence time of 15 min to 45 min at a temperature of 110° C. to 150° C.

15. The method of claim 1, further comprising a step of removing unreacted diisocyanate monomer from the reaction solution after completion of the polymerization reaction;
wherein the removing of the unreacted diisocyanate monomer is carried out by means of evaporation method, and the evaporation method is any one selected from the group consisting of thin film evaporation method, falling film evaporation method, short range evaporation method, and reduced pressure rectification method.
